(19) United States
(12) Patent Application Publication
(10) Pub. No.: US 2003/0211528 A1
(43) Pub. Date: Nov. 13, 2003

(54) Exponential amplification of sub-picogram nucleic acid samples with retention of quantitative representation

(76) Inventor: Norman Iscove, Toronto (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/386,103

(22) Filed: Mar. 12, 2003

Related U.S. Application Data
(60) Provisional application No. 60/363,254, filed on Mar. 12, 2002.

(51) Int. Cl. C12Q 1/68; C12P 19/34

(52) U.S. Cl. 435/6; 435/91.2

(57) ABSTRACT

Within the near future it will be possible to survey expression of all genes in a sample by microarray analysis. Current methods for nucleic acid amplification require microgram amounts of complementary DNA or RNA for hybridization to microarrays. Without amplification, such amounts are only obtainable from millions of cells. However, frequently such numbers are not available: aspiration biopsies, rare population subsets isolated by cell sorting or laser capture, or micromanipulated single cells are examples where few or even only single cells containing the desired information may be at hand. The current invention reduces the input amount of RNA needed for microarray analysis by a million-fold, and yields reproducible results from the picogram range of total RNA obtainable from a single cell. Of central importance to the present claims, the invention generates an amplified cDNA product in which the abundance relationships of the original RNA are faithfully preserved throughout amplification.

### CROSS REFERENCE TO RELATED APPLICATION

This application claims priority from U.S. provisional application No. 60/363,254 filed on Mar. 12, 2002, which is incorporated by reference in its entirety.

### FIELD OF THE INVENTION

The invention relates to a method for the exponential amplification of sub-picogram nucleic acid samples with retention of quantitative representation.

### BACKGROUND OF THE INVENTION

Within the near future it will be possible to survey expression of all genes in a sample by microarray analysis. Current methods require microgram amounts of complementary DNA or RNA for hybridization to microarrays. Without amplification, such amounts are only obtainable from millions of cells. However, frequently such numbers are not available: aspiration biopsies, rare population subsets isolated by cell sorting or laser capture, or micromanipulated single cells are examples where few or even only a single cell containing the desired information may be at hand. Methods for nucleic acid amplification could be used in such instances to generate the required amounts. The difficulty is to obtain amplification without distorting the quantitative relationships between different transcripts in the original sample. Linear isothermal RNA amplification has been shown to be capable of expanding cDNA as much as 1000-fold while still preserving the original abundance relationships of individual messages^{1, 2}. However, the available procedures are complex, labor-intensive and time consuming³. More significantly, abundance information degrades unacceptably when amplification is pushed beyond 1000-fold, effectively limiting the application of such protocols to amounts of RNA available from at least 1000 to 10000 cells². Exponential amplification, on the other hand, is a relatively simple technology, but is universally considered to introduce an even greater and unacceptable bias into abundance relationships. Baugh et al (2001), Phillips et al (2000), Freeman et al (2000) and Dixon et al (2000) propagate the view that PCR-based methods for universal amplification can not be used for quantitative purposes. These constraints have placed beyond the range of current methodology the secure and routine application of microarray analysis to RNA isolated from a single cell.

### SUMMARY OF THE INVENTION

The current invention is a highly optimized, rapid and relatively simple procedure for exponential amplification of nucleic acids. In one embodiment, the exponential amplification is global RT-PCR amplification of nucleic acid, such as mRNA or cDNA. One strong advantage of the current invention is that it reduces the starting amount of RNA needed for microarray analysis by a million-fold, and yields reproducible results from the picogram range of total RNA, which is obtainable from a single cell. In a particularly preferred embodiment, the amount of nucleic acid is sub-picogram and is optionally obtained by microneedle withdrawal of sample from a cell, such as a sample of cell cytoplasm. The volume removed by a microneedle may be as low as 1 picolitre and contain at little as 1 picogram of DNA or RNA. Importantly, the invention generates an exponentially amplified cDNA product in which the abundance relationships of the original RNA are faithfully preserved throughout amplification as high as 3×10¹¹-fold. The present invention is directed to methods for generating nucleic acid targets and probes preferably for use in gene expression monitoring systems. The method optionally comprises (A) providing polyadenylated mRNA, (B) preparing a first oligonucleotide primer, (C) contacting the mRNA with the first primer to generate by DNA polymerase or reverse transcriptase reaction from the polyadenylated mRNA, DNA strands that are substantially complementary to the polyadenylated mRNA, (D) adding a polynucelotide tail to the 3′ end of the DNA strands whereby the DNA strands have a first portion that is complementary to the polyadenylated mRNA and a tail portion, (E) preparing a second oligonucleotide primer, (F) contacting the complementary DNA with the second primers to generate by a DNA polymerase reaction from the tailed DNA, DNA strands that are substantially complementary to the tailed DNA; and (G) contacting the DNA strands with the first primer and the second primer to amplify exponentially by at least 1000-fold the DNA strands by repetitive cycles of thermal denaturation followed by DNA polymerase reaction.

In one embodiment residual RNA remaining after step C is digested by addition of RNAse H.

In one embodiment the reverse transcription reaction occurs between about 30° C. to about 100° C., and the DNA polymerase reaction occurs between about 23° C. to about 100° C.

In one embodiment there are at least 20 repetitive cycles of thermal denaturation, annealing, and DNA polymerase reaction.

In one embodiment the repetitive cycles occur between 23° C. to 100° C., denaturation occurs between 90° C. to 100° C., annealing occurs between 37° C. to about 75° C., and DNA polymerase reaction occurs between about 37° C. to about 80° C.

In one embodiment nucleotides that are covalently coupled to fluorochromes, are incorporated during the repetitive cycles of thermal denaturation, annealing and DNA polymerase reaction to directly generate fluorochrome-coupled nucleic acid targets or probes. In another embodiment nucleotides that contain reactive side groups are incorporated during the repetitive cycles of thermal denaturation, annealing and DNA polymerase reaction to directly generate reactive-side-group-coupled nucleic acid targets or probes. In yet another embodiment the reactive side groups of the reactive-side-group-coupled nucleic acid targets or probes interact with fluorochromes to generate fluorochrome-coupled nucleic acid targets or probes.

The first oligonucleotide primer has a first segment with a unique sequence and a second segment that is substantially complementary to the polyadenylated mRNA and capable of template-dependent first strand synthesis. The second oligonucleotide primer has a first segment that contains a unique sequence and a second segment that is substantially complementary to the tail portion of the DNA strand and capable of template-dependent second strand synthesis.

In one embodiment the first and second oligonucleotide primers are identical. In another embodiment the oligonucleotide primers are different.

In one embodiment the first and second oligonucleotide primers provide for non-directional amplification of the polyadenylated mRNA. In another embodiment the first and second oligonucleotide primers provide for directional amplification of the polyadenylated mRNA.

In another embodiment the first and second oligonucleotide primers are anchored primers. In yet another embodiment the anchoring primers contain anchoring nucleotides selected from the group consisting of adenine, guanine, cysteine and thymidine.

In one embodiment the RNA is isolated from a biological sample selected from the group consisting of a body fluid, stool, a single cell, dissected tissue, microdissected tissue, a tissue subregion, a tissue biopsy sample, cells recovered from body fluids or from the body in aspirates or scrapings or washings, a cell sorted population and a cell culture. In another embodiment the RNA is isolated from a cell or tissue selected from the group consisting of brain, liver, heart, kidney, lung, spleen, eye, retina, bone, lymph node, endocrine, endocrine gland, secretory gland, reproductive organ, blood, marrow, bone, cartilage, muscle, fat, connective tissue, nerve, vascular tissue, skin, hair, epithelial and mesothelial structures or surfaces. In yet another embodiment the RNA is isolated from a cell or tissue selected from the group consisting of embryonic, pathological and tumorigenic.

In one embodiment the RNA is present in an amount less than 10 ng, more preferably between 0.1 picograms and 10 ng of RNA, and the polynucleotide tail is poly(A), poly(G), poly(C) or poly(T). The mRNA is optionally obtained from a single cell, for example by extracting, lysing or homogenizing the full cell or taking an extract from the cell. Preferably the amplified nucleic acid is limited to all or part of the 200 to 600 nucleotides at the 3′ terminus of the mRNA.

In one embodiment the gene expression monitoring system is selected from the group comprising DNA array, biochip, DNA chip, DNA microarray, gene array, real time quantitative PCR and any device or method designed to measure quantitatively the representation of one or more sequences in the amplified sample. The methods of the invention thus optionally further include the additional step of quantitatively measuring the representation of one or more nucleic acids in the amplified sample, preferably by one of the above methods to obtain a numerical value for relative abundance. For example, one could determine whether one or more genes is amplified in a tumor sample.

The present invention also includes probes and targets generated by any of the processes described above. The invention also includes the use of the process of the invention to generate cDNA libraries for the purpose of measuring the representation of particular gene transcripts.

According to another aspect of the present invention there is provided a kit useful for generating nucleic acid probes for use in gene expression monitoring systems. in another aspect of the present invention there is provided a kit useful for generating nucleic acid targets for use in gene expression monitoring systems. The components of the kit may include a reverse transcriptase, a DNA polymerase, a terminal deoxynucleotidyl transferase, oligonucleotide primers and/or additional reagents (e.g. enabling reagents) useful in labeling the targets and probes of the current invention, such as a buffer. The kit may also contain reagents for use in generating cDNA libraries.

In another embodiment, the invention relates to a process for generating nucleic acid targets, for example, for use in a gene expression monitoring, and may optionally comprise the steps of:

(A) providing an RNA preparation (ie. any RNA source) that comprises polyadenylated mRNA;

(B) providing a first oligonucleotide primer that comprises

(1) a first segment containing a unique sequence (ie. arbitrary sequence) lacking internal nucleotide similarity that promotes self annealing (so that the first segment will not anneal to other first segments); and

(2) a second segment being substantially complementary to the polyadenylated mRNA and capable of template-dependent first strand synthesis;

(C) contacting the mRNA with the first primer, preferably at reaction temperature (which means that segments do not contact the enzyme except at the reaction temperature (ie. the primer does not contact the enzyme and template except at the reaction temperature; also known as a hot start), which is preferably at 37-60° C., 50-60° C., or more preferably 50° C.). This reaction generates by DNA polymerase or reverse transcriptase reaction from the polyadenylated mRNA, DNA strands that are substantially complementary to the polyadenylated mRNA, using a reverse transcriptase lacking RNAse H activity;

(D) digestion of the RNA, for example by digestion with RNAase H;

(E) adding a polynucleotide tail to the 3′ end of the DNA strands whereby the DNA strands have a first portion that is complementary to the polyadenylated mRNA and a tail portion;

(F) providing a second oligonucleotide primer that comprises

(1) a first segment containing a unique sequence (ie. arbitrary sequence) lacking internal nucleotide similarity that promotes self annealing (so that the first segment will not anneal to other first segments); and

(2) a second segment being substantially complementary to the tail portion of the DNA strand and capable of template-dependent second strand synthesis;

(G) contacting the complementary DNA with the second primer to generate by a DNA polymerase reaction from the tailed DNA, DNA strands that are substantially complementary to the tailed DNA at reaction temperature which is preferably at 37-60° C., 50-60° C., or more preferably 50° C.; and

(H) contacting the DNA strands with the first primer and the second primer to amplify exponentially, by at least 1000-fold, the DNA strands by repetitive cycles of thermal denaturation, annealing and DNA polymerase reaction with retention of quantitative representation of the DNA strands. This is preferably done at a higher temperature than at the preceding step (G), in the temperature range of 37° C.-72° C., 55-66° C. or more preferably 60° C.

One or more of the above steps may be varied or used with other methods of the invention.

Other features and advantages of the present invention will become apparent from the following detailed description It should be understood, however, that the detailed description and the specific examples while indicating preferred embodiments of The invention are given by way of illustration only, since various changes and modifications within the spirit and scope of the invention will become apparent to those skilled in the art from this detailed description.

### BRIEF DESCRIPTION OF THE DRAWINGS

Preferred embodiments of the invention will be described in relation to the figures in which:

FIG. 1. Agarose gel electrophoresis and southern blotting of cDNA globally amplified from varying amounts of total HeLa cell or Universal Human Reference RNA. Upper panels show ethidium bromide stained gels, lower panels show blots hybridized with a radiolabelled probe for ribosomal L27. As shown, similar amounts of cDNA were generated from each starting amount of RNA indicated, suggesting that amplification proceeded essentially to saturation in each case.

FIG. 2. Scatter plots testing reproducibility of normalized hybridization intensities from unamplified (“Direct”) or amplified (“Amp”) targets on microarrays in independent experiments. The average relative error (RE) for the pairwise comparisons is indicated on each panel. A and C. Correspondence between intensities of duplicate spots on an array hybridized with unamplified or amplified target respectively. All Cy3 and Cy5 data were included. B, D and E. Correspondence between hybridization intensities on corresponding individual spots on paired arrays hybridized to independently prepared unamplified or amplified targets respectively. Results from independent amplifications from 10 or 1 ng total RNA are compared in D, and from 10 ng or 10 pg in E.

FIG. 3. Scatter plots showing the relationship between hybridization intensities from HeLa cell versus Universal Human Reference cDNA targets before and after global amplification. Series I. Plots of HeLa versus Universal Human Reference hybridization intensities on individual array spots. The two plots on the left show spot intensities after hybridization to independently prepared unamplified targets in separate hybridization experiments. The next four plots show results from amplified targets prepared from differing amounts of total RNA. All patterns consist of a main set of spots on the diagonal representing equivalent intensities developed from HeLa and Universal Reference targets, and a subset of spots which hybridize HeLa to a lesser degree than Universal Reference target cDNA. Series II. A and B. Frequency histograms of the HeLa/Universal Reference spot intensity ratios obtained in the array hybridization illustrated in Set I, left panel. A, distribution of ratios from spots whose hybridization intensities fell beneath the cutoff defined in Procedures. B, ratios from spots with hybridization intensities above the rejection threshold. C-G. Correspondence between HeLa/Universal Reference intensity ratios obtained in independent hybridizations for corresponding array spots. The mean relative errors for the pairwise spot comparisons are indicated beneath each plot, along with the slope of the straight line fitted by least squares to the logarithms of the ratio values. C. Ratios obtained after hybridization of independently prepared unamplified cDNA targets to an array pair. D and E. Ratios obtained from cDNA targets independently amplified from the indicated amounts of total RNA. F and G. Direct comparison of ratios obtained from amplified targets and unamplified targets on independent arrays.

FIG. 4. Non-directional amplification. Preferably both cDNA ends contain complete primer sequences after step 3, and other preferable steps are as follows:

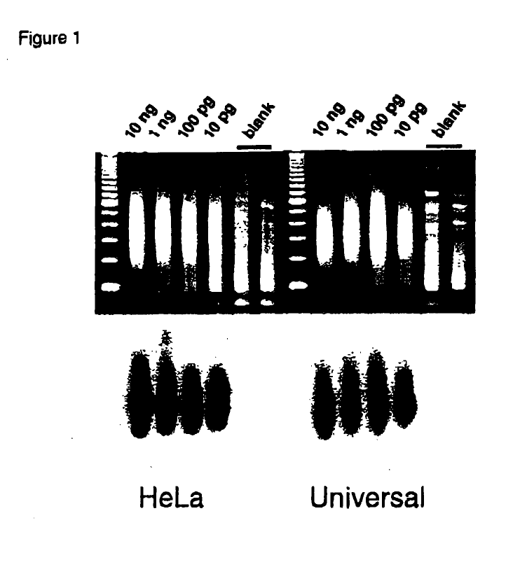

1. The first strand synthesis is primed at 50 deg, by “hot start,” minimizing false priming at inappropriate sites and especially minimizing priming on DNA rather than mRNA templates;

2. The first strand is primed with complete primer, i.e. T(24) plus the unique heel, thus defining one transcript end;

3. First strand synthesis is executed with a reverse transcriptase mutant lacking RNAse H activity, increasing the amount of first cDNA strand generated;

4. Residual RNA is digested by addition of RNAse H after completion of first strand synthesis, increasing the yield of the tailing step (2) in the figure;

5. The primer is allowed to anneal to the first strand at a lower temperature 50° C. for generation of the second strand (step (3) in the figure), increasing the efficiency of second strand generation. This change, and the use of complete primer to generate the first strand, Together eliminate the need for extended times and lower stringency in the ensuing amplification cycles that were required in the original protocol, and therefore further reduce opportunities for mispriming;

6. The primer is different form the primer originally described, and shows greatly reduced tendency to self-interact to generate primer amplification artifact;

7. The entire procedure is completed quickly compared to conventional processes (e.g. half the time);

8. Libraries generated by the improved procedure consist of a higher proportion of clones containing valid 3′ UTR sequence and a polyadenylation signal, validating the improved resistance of the protocol to mispriming on templates other than polyA.

FIG. 5. Directional amplification preserves the orientation of the original transcript by the use of upstream and downstream primers (1 and 2 in the Figure) that differ from each other. The first and second strands are generate at 40 and 50 degrees respectively, based on hybridization of the T(15) tracts. The primers can initiate synthesis only from a single unique position by virtue of an anchoring nucleotide (N in the Figure, representing an A, G or C). Incorporation of the anchoring nucleotide significantly reduces the generation of primer interaction artifacts. Once the two priming sites are in position at the transcript ends after step (3), amplification is performed at 60 or 65 degrees, where T(15) hybridization is insufficient to prime new strand synthesis, ensuring that the two transcript ends remain unique.

### DETAILED DESCRIPTION OF THE PREFERRED EMBODIMENTS

Definitions

A probe is defined as a nucleic acid, preferably a synthetic oligonucleotide or a cDNA with known identity. A target is defined as a sample, such as a labeled sample. In other words, a probe is the tethered nucleic acid with known sequence, whereas a target is the free nucleic acid sample comprised of multiple nucleic acids, such as cDNAs, whose abundances are being measured.

An anchored primer is constructed with one or more extra nucleotides in the 3′-end. The extra nucleotide(s) will permit amplification only if the primer binds to the 5′-end of the primer's complementary sequence on the template nucleic acid. Such extra nucleotides are termed anchors and they assure that amplification will always start from a single unique position on the template. Anchoring primers therefore enhance the precision and specificity of the amplification reaction.

Template RNA

The template RNA may be any ribonucleic acid of interest, known or unknown to the practitioner. Template RNA may be artificially synthesized or isolated from natural sources Preferably the RNA is polyadenylated RNA. More preferably the polyadenylated RNA is biologically active or encodes a biologically active polypeptide.

The RNA of the present invention may be obtained or derived from any tissue or cell source. In a preferred embodiment the RNA is isolated from a single cell source. It may be an entire single cell extract or a microneedle withdrawal of cytoplasm from a single cell. The RNA to be amplified may be obtained from any biological or environmental source, including animal, plant, virus, bacterium, fungus, or algae, or from any sample, including body fluid or soil. In one embodiment, eukaryotic tissue is preferred, and in another, mammalian tissue is preferred, and in yet another, human tissue is preferred. The tissue or cell source may include a tissue biopsy sample, a body fluid, stool, dissected tissue, microdissected tissue, a tissue subregion and cells recovered from body fluids or cells recovered from the body in aspirates or scrapings or washings, a cell sorted population, cell culture, or a single cell. In a most preferred embodiment the RNA is polyadenylated RNA isolated from a single cell.

In a preferred embodiment, the tissue source may include brain, liver, heart, kidney, lung, spleen, eye, retina, bone, lymph node, endocrine, endocrine gland, secretory gland, reproductive organ, sensory organ, blood, marrow, cartilage, muscle, fat, connective tissue, nerve, vascular tissue, skin, hair, and epithelial and mesothelial structures or surfaces. In yet another preferred embodiment, the tissue or cell source may be normal, non-embryonic, embryonic, pathological or tumorigenic.

Tumorigenic tissue according to the present invention may include tissue associated with malignant and pre-neoplastic conditions, not limited to the following: acute lymphocytic leukemia, acute myelocytic leukemia, myeloblastic leukemia, promyelocytic leukemia, myelomonocytic leukemia, monocytic leukemia, erythroleukemia, chronic myelocytic (granulocytic) leukemia, chronic lymphocytic leukemia polycythemia vera, lymphoma, Hodgkin's disease, non-Hodgkin's disease, multiple myeloma, Waldenstrom's macroglobulinemia, heavy chain disease, solid tumors, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

The amount of total cellular RNA used for amplification may be 10 ng, preferably less than 10 ng, and most preferably between 10 picograms and 10 ng corresponding to a range of mRNA between 0.2 pg and 0.2 ng. In mammalian cells, polyadenylated mRNA is about 3% of total cellular RNA. mRNAs naturally range in length from 300 bases to several thousand bases in length. The methods of the invention preferably capture and amplify the 200-600 nucleotides at the 3′ terminus of a nucleic acid, more preferably the 200-500 nucleotides at the 3′terminus.

Reverse Transcriptase-Polymerase Chain Reaction (RT-PCR)

RT-PCR is a methodology well known in the art, but universally was, before this invention, considered to be unsuitable for adaptation to the purposes of measurement of relative abundances of large numbers of transcripts (Baugh et al (2001), Phillips et al (2000), Freeman et al (2000) and Dixon et al (2000)). The invention provides a method for global RT-PCR to enhance sensitivity, and to address deficiencies which could impact on fidelity and sensitivity, including amplification of primer concatemers or of sequences lacking polyadenylation signals which originated from mispriming on either RNA or genomic DNA templates. The invention is more robust and inclusive when applied to single cells, and generates libraries from single cells in which 72% of clones (vs<30% before modification) contain polyadenylation signals. Major improvements include introduction of hot start conditions for initiating the reverse transcriptase reaction, use of a mutant reverse transcriptase lacking RNAse H activity, degredation of residual RNA with RNAse H at the end of reverse transcription, redesign of the poly(T) primer, introduction of a single end-conversion step, and amplification at more stringent annealing temperature. RT-PCR is a methodology well known in the art, but universally considered to be unsuitable for adaptation to the purposes of measurement of relative abundances of large numbers of transcripts. In one embodiment of the invention using RT-PCR, the reaction mixture is first incubated in an appropriate buffering agent at a temperature sufficient to allow synthesis of a DNA molecule complementary to at least a portion of typically one or a small number of different RNA templates. After reverse transcription of an RNA template to produce a cDNA molecule, the cDNA is incubated in an appropriate buffering agent under conditions sufficient for replication of the cDNA molecule. The reaction mixture may be the same as that of the previous reverse transcription reaction mixture, as employed in coupled (also called continuous, or one-step) RT-PCR, or the reaction mixture may comprise an aliquot of the previous reverse transcription reaction mixture and may be further modified for nucleic acid amplification, as in uncoupled (or two-step) RT-PCR. Components of a replication reaction mixture typically include a nucleic acid template (in this instance the cDNA); a nucleic acid polymerase; and the appropriate nucleotide building blocks needed for nucleic acid synthesis. Nucleic acid amplification refers to the polymerization of a nucleic acid whose sequence is determined by, and complementary to, another nucleic acid. Preferably DNA amplification occurs repetitively, thus replicating both strands of the nucleic acid sequence, i.e., DNA complementary to the RNA template, and DNA whose nucleic acid sequence is substantially identical to the RNA template. Repetitive, or cyclic, DNA amplification may be advantageously accomplished using a thermostable polymerase in a DNA polymerase reaction.

In one embodiment the DNA amplification is linear amplification. In a more preferred embodiment DNA is amplified exponentially by repetitive cycles of thermal denaturation followed by DNA polymerase reaction. In another preferred embodiment the DNA strands are amplified exponentially by at least 1×10³. In another preferred embodiment the DNA strands are amplified exponentially by at least 1×10⁴. In another preferred embodiment the DNA strands are amplified exponentially by at least 1×10⁵. In another preferred embodiment the DNA strands are amplified exponentially by at least 1×10⁶ In another preferred embodiment the DNA strands are amplified exponentially by at least 1×10⁷. In another preferred embodiment the DNA strands are amplified exponentially by at least 1×10⁸. In another preferred embodiment the DNA strands are amplified exponentially by at least 1×10⁹. In another preferred embodiment the DNA strands are amplified exponentially by at least 1×10¹⁰. In another preferred embodiment the DNA strands are amplified exponentially by at least 1×10¹¹. In another preferred embodiment the DNA strands are amplified exponentially by at least 3×10¹¹.

In one preferred embodiment there is at least 20 repetitive cycles of exponential DNA amplification. In another embodiment there is at least 30 repetitive cycles of exponential DNA amplification. In yet another embodiment there is at least 40 repetitive cycles of exponential DNA amplification. In yet another embodiment there is at least 50 repetitive cycles of exponential DNA amplification. In yet another embodiment there is at least 60 repetitive cycles of exponential DNA amplification. In yet another embodiment there is at least 65 repetitive cycles of exponential DNA amplification.

Reverse Transcriptase Reaction

Components of a reverse transcription reaction mixture typically include an RNA template, from which the complementary DNA (cDNA) is transcribed; a nucleic acid polymerase that exhibits reverse transcriptase activity; and the appropriate nucleotide bases necessary for nucleic acid synthesis. For the purposes of this invention, cDNA is defined as any DNA molecule whose nucleic acid sequence is complementary to an RNA molecule. An RNA template is defined as any RNA molecule used to provide a nucleic acid sequence from which a cDNA molecule may be synthesized. The synthesis of cDNA from an RNA template is typically accomplished by utilizing a nucleic acid polymerase that exhibits reverse transcriptase activity. For the purposes of this invention, reverse transcriptase activity refers to the ability of an enzyme to polymerize a cDNA molecule from an RNA template, and reverse transcriptase broadly refers to any enzyme possessing reverse transcriptase activity. Reverse transcription typically occurs in a temperature range from about 20° C. to about 75° C., preferably from about 35° C. to about 70° C., more preferably at 50° C.

Tailing

In a preferred embodiment of the present invention, the single-stranded cDNA produced using a mRNA population as template may be liberated from any resulting RNA:DNA heteroduplexes by heat or enzyme treatment (e.g., RNase H to digest the RNA template). Terminal deoxynucleotidyl transferase may be used to add poly(A) or poly(G) sequences to the 3′-termini of the single-stranded DNA to provide a priming site for a DNA polymerase reaction. The double-stranded DNA of the present invention may Then be synthesized from the heterogeneous single-stranded DNA.

In a preferred embodiment of the present invention, the ends of the double-stranded DNA may be blunted to prevent any concatenation of the double-stranded DNA. T4 DNA polymerase or Escherichia coli DNA polymerase I (Klenow fragment), for example, may be used preferably To produce blunt ends in the presence of the appropriate dNTPs.

Polymerase Chain Reaction

The preferred method for amplifying DNA in the instant specification is the Polymerase Chain Reaction (PCR). PCR is a technique well known in the art. Polymerase Chain Reaction is used to amplify DNA by subjecting a reaction mixture to cycles of (I) Thermal denaturation, (II) oligonucleotide primer annealing, and (III) DNA polymerase reaction.

In one embodiment the DNA is amplified linearly, more preferably the DNA is amplified exponentially.

Preferred reaction conditions for amplification comprise thermocycling, i.e., alternating the temperature of the reaction mixture to facilitate each of the steps of the cycle. The reaction mixture is typically extended through multiple cycles of denaturation, annealing and DNA polymerase reaction, augmented (optionally and preferably) with an initial prolonged denaturation step and a final prolonged extension (polymerization) step. Thermocycling typically occurs within a temperature range of between about 23° C. to about 100° C., and preferably between about 37° C. to about 95° C. Nucleic acid denaturation typically occurs between about 90° C. about 100° C., preferably about 94° C. Annealing typically occurs between about 37° C. to about 75° C. preferably about 60° C. DNA polymerase reaction typically occurs between about 55 to about 80° C., preferably between about 65° C. to about 75° C., more preferably at 72° C. The number of PCR cycles varies immensely, depending upon practitioner preference and the quantity of DNA product desired. Preferably, the number of PCR cycles in a reaction ranges from about 5 to about 99, most preferably about 30 or 35 cycles in the instant specification.

Components of a PCR reaction mixture typically include a DNA template, from which the complementary DNA is amplified; a nucleic acid polymerase that exhibits DNA polymerase activity; and the appropriate nucleotide bases necessary for nucleic acid synthesis

Reverse Transcriptases

Reverse transcriptases useful in the present invention may be any polymerase that exhibits reverse transcriptase activity. Preferred enzymes include those that exhibit reduced RNase H activity. Several reverse transcriptases are known in the art and are commercially available (e.g., from Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.) Preferred reverse transcriptases include: Avian Myeloblastosis Virus reverse transcriptase (AMV-RT), Moloney Murine Leukemia Virus reverse transcriptase (MMLV-RT), Human Immunovirus reverse transcriptase (HIV-RT), EIAV-RT, RAV2-RT, C. hydrogenoformans DNA Polymerase, rTth DNA polymerase, SUPERSCRIPT I, SUPERSCRIPT II, and mutants, variants and derivatives thereof It is to be understood that a variety of reverse transcriptases may be used in the present invention, including reverse transcriptases not specifically disclosed above, without departing from the scope or preferred embodiments thereof.

DNA Polymerases

DNA polymerases useful in the present invention may be any polymerase capable of replicating a DNA molecule. Preferred DNA polymerases are thermostable polymerases, which are especially useful in PCR. Thermostable polymerases are isolated from a wide variety of thermophilic bacteria, such as Thermus aquaticus (Faq), Thermus brockianus(Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritime (Tma), and other species of the Thermotoga genus, Pyrococcus furiosus (Pfu), Pyrococcus woesei (Pwo) and other species of the Pyrococcus genus, Bacillus sterothermophilus (Bst), Sulfolobus acidocaldarius (Sac) Sulfolobus solfataricus (Sso), Pyrodictium occulrum (Poc), Pyrodictium abyssi (Pab), and Methanobacterium thermoauotrophicum (Mth), and mutants, variants or derivatives thereof. Several DNA polymerases are known in the art and are commercially available (e.g., from Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.) Preferably the thermostable DNA polymerase is selected from the group of Taq, Thr, Tfl, Tru, Tth, Tli, Tac, Tne, Tma, Tih, Tfi, Pfu, Pwo, Kod, Bst, Sac, Sso, Poc, Pab, Mth, Pho, ES4 VENT.™, DEEPVENT.™, and active mutants, variants and derivatives thereof. It is to be understood that a variety of DNA polymerases may be used in the present invention, including combinations of DNA polymerases, also admix of polymerases with 3′ proofreading activity, and also including DNA polymerases not specifically disclosed above, without departing from the scope or preferred embodiments thereof.

Oligonucleotide Primers

Oligonucleotide primers are oligonucleotides used to hybridize to a region of a transcript to facilitate the polymerization of a complementary nucleic acid. In preferred RT-PCR techniques, primers serve to facilitate reverse transcription of a first nucleic acid molecule complementary to a potion of an RNA template (e.g., a cDNA molecule), and also to facilitate replication of the nucleic acid (e.g., PCR amplification of DNA). Oligonucleotide primers useful in the present invention may be any oligonucleotide of two or more nucleotides in length. Preferably, PCR primers are about 15 to about 30 bases in length, and are not palindromic (self-complementary) or complementary to other primers that may be used in the reaction mire. Primers may be, but are not limited to, random primers, homopolymers, or primers specific to a target RNA template (e.g., a sequence specific primer), more preferably the primers are anchored primers.

An oligonucleotide primer may be applied to the poly(A), poly(G), poly(C) or poly(T) failed heterogeneous single-stranded DNA. The oligonucleotide primer preferably includes a poly(T) or poly(C) region complementary the poly(A) or poly(G) tail attached to the single-stranded DNA. In addition, the oligonucleotide primer preferably contains a unique sequence that is not complementary to the poly(A) or poly(G) tail.

Any primer may be synthesized by a practitioner of ordinary skill in the an or may be purchased from any of a number of commercial venders (e.g., from Boehringer Mannheim Corp., Indianapolis, Ind.; New England Biolabs, Inc., Beverley, Mass.; Pharmacia LKB Biotechnology, Inc., Piscataway, N.J.). It is To be understood that a vast array of primers may be useful in the present invention, including those not specifically disclosed herein, without departing from the scope or preferred embodiments thereof.

Nucleotide Bases

Nucleotide bases useful in the present invention may be any nucleotide useful in the polymerization of a nucleic acid. Nucleotides may be naturally occurring, unusual, modified, derivative, or artificial. Nucleotides may be unlabeled, or detectably labeled by methods known in the art (e.g., using radioisotopes, vitamins, fluorescent or chemiluminescent moieties, digoxigenin). Preferably the nucleotides are deoxynucleoside triphosphates, dNTPs (e.g., dATP, dCTP, dGTP, dTTP, dITP, dUTP, .alpha.-thio-dNITs, biotin-dUTP, fluorescein-dUTP, digoxigenin-dUTP, 7-deaza-dGTP) dNTPs are also well known in the art and are commercially available from vendors (e.g., from Boehringer Mannheim Corp., Indianapolis, Ind.; New England Biolabs, Inc., Beverley, Mass.; Pharmacia LKB Biotechnology, Inc., Piscataway, N.J.).

Labelling cDNA

In a preferred embodiment of the present invention, the multiple copies of cDNA may be labeled by the incorporation of biotinylated, fluorescently labeled or radiolabeled CTP during the cDNA synthesis. Alternatively, labeling of the multiple copies of cDNA may occur following the cDNA synthesis via the attachment of a detectable label in the presence of terminal transferase. In a preferred embodiment of the present invention, the detectable label may be radioactive, fluorometric, enzymatic, or calorimetric, or a substrate for detection (e.g., biotin). Other detection methods, involving characteristics such as scattering, IR, polarization, mass, and charge changes, may also be within the scope of the present invention.

Gene Expression Monitoring Systems

In a preferred embodiment, the amplified cDNA of the present invention may be analyzed with a gene expression monitoring system. A gene expression monitoring system according to the present invention may be a nucleic acid probe array such as the GeneChip.RTM. nucleic acid probe array (Affymetrix, Santa Clara, Calif.). A nucleic acid probe array preferably comprises nucleic acids bound to a substrate in known locations In other embodiments, the system may include a solid support or substrate, such as a membrane, filter, microscope slide, microwell, sample tube, bead, bead array, or the like. The solid support may be made of various materials, including paper, cellulose, nylon, polystyrene, polycarbonate, plastics, glass, ceramic, stainless steel, or the like. The solid support may preferably have a rigid or semi-rigid surface, and may preferably be spherical (e.g., bead) or substantially planar (e.g. flat surface) with appropriate wells, raised regions, etched trenches, or the like. The solid support may also include a gel or matrix in which nucleic acids may be embedded.

The gene expression monitoring system, in a preferred embodiment, may comprise a nucleic acid probe array (including an oligonucleotide array, a cDNA array, a spotted array, and the like), membrane blot (such as used in hybridization analysis such as Northern, Southern, dot, and the like), real time PCR, or microwells, sample rubes, beads or fibers (or any solid support comprising bound nucleic acids). The gene expression monitoring system may also comprise nucleic acid probes in solution.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue. In a preferred embodiment, the proportional amplification methods of the present invention can provide reproducible results (i.e., within statistically significant margins of error or degrees of confidence) sufficient to facilitate the measurement of quantitative as well as qualitative differences in the tested samples. The proportional amplification methods of the present invention may also facilitate the measurement of the abundance of transcripts containing specific mutations that can serve, for example, as markers of neoplastic cells in samples of limited size.

Nucleic Acid Detection System

In yet another preferred embodiment of the present invention, a nucleic acid detection system, the proportionally amplified cDNA or fragments thereof, may be immobilized directly or indirectly to a solid support or substrate by methods known in the art (e.g., by chemical or photoreactive interaction, or a combination thereof). The resulting immobilized cDNA may be used as probes to detect nucleic acids in a sample population that can hybridize under desired stringency conditions. Such nucleic acids may include DNA contained in the clones and vectors of cDNA libraries

Kits for Facilitating the Measurement of Gene Expression in Small Samples of Cells or RNA

The materials for use in the present invention are ideally suited for the preparation of a kit suitable for the single-phase proportional amplification of nucleic acids. Such a kit may comprise reaction vessels, each with one or more of the various reagents, preferably in concentrated form, utilized in the methods. The reagents may comprise, but are not limited to the following: low modified salt buffer, appropriate nucleotide triphosphates (e.g. dATP, dCTP, dGTP, dTTP; or rATP, rCTP, rGTP, and UTP) reverse transcriptase, RNase H, terminal deoxynucleotidyl transferase, thermostable DNA polymerase, RNA polymerase, and the appropriate primer complexes. Such kits may also include materials and reagents suitable for nucleic acid purifications, for example purification columns, and reagents and materials for generating labelled cDNA. In addition, the reaction vessels in the kit may comprise 0.2-1.0 ml tubes capable of fitting a standard PCR thermocycler, which may be available singly, in strips of 8, 12, 24, 48, or 96 well plates depending on the quantity of reactions desired. Hence, the single-phase amplification of nucleic acids may be automated, e.g., performed in a PCR theromcycler. The PCR thermocyclers may include, but are not limited to the following: Perkin Elmer 9600, MJ Research PTC 200, Techne Gene E, Erichrom, and Whatman Biometra T1 Thermocycler.

Also, the automated machine of the present invention may include an integrated reaction device and a robotic delivery system. In such cases, part of all of the operation steps may automatically be done in an automated cartridge.

Without further elaboration, one skilled in the an with the preceding description can utilize the present invention to its fullest extent. The following examples are illustrative only, and not intended to limit the remainder of the disclosure in any way.

It will be readily apparent to one of ordinary skill in the art that other suitable modifications and adaptations of the compositions and methods of the invention described herein are obvious and may be made without departing from the scope of the invention or the disclosed embodiments thereof. Having now described the present invention in detail, the same will be more clearly understood by reference to the following examples, which are included herewith for purposes of illustration only and are not intended to be limiting of the invention.

Buffering Agents and Salt Solutions

Buffering agents and salts useful in the present invention provide appropriate stable pH and ionic conditions for nucleic acid synthesis, e.g., for reverse transcriptase and DNA polymerase activity. A wide variety of buffers and salt solutions and modified buffers are known in the art that may be useful in the present invention, including agents not specifically disclosed herein. Preferred buffering agents include, but are not limited to, TRIS, salts of cacodylic acid, salts of acetic acid, carbonate or bicarbonate salts, TRICINE, BIS-TRICINE, HEPES, MOPS, TES, TAPS, PIPES, CAPS. Preferred salt solutions include, but are not limited to solutions of; potassium acetate, potassium sulfate, ammonium sulfate, ammonium chloride, ammonium acetate, magnesium chloride, magnesium acetate, magnesium sulfate, manganese chloride, manganese acetate, manganese sulfate, sodium chloride, sodium acetate, lithium chloride, lithium acetate, and ionic cobalt for example in the form of cobalt chloride Other constituents potentially useful in the present invention include metal chelating agents such as EDTA; antioxidants such as dithiothreitol; saccharides or polysaccharides with protein stabilizing activity; and agents which enhance the interactions of nucleic acids such as suitable salts and polyethylene glycol.

### EXAMPLES

Independently Prepared Amplified Targets Yield Similar Results on Microarrays

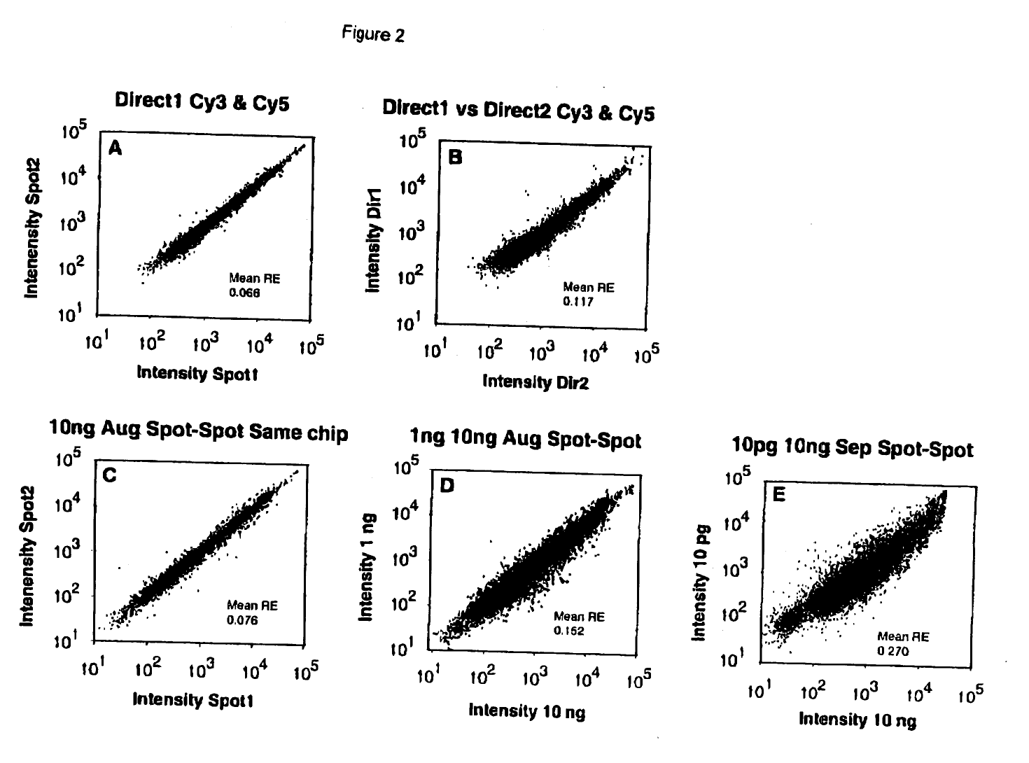

FIG. 2 shows a series of scatter plots that measure the reproducibility of hybridization intensities obtained from independent preparations of unamplified (“Direct”) or amplified cDNA, always from the same archival HeLa and Reference RNA source. Every Cy3 and Cy5 fluorescence intensity on an array spot is represented by a point in the plots. Panels A and C illustrates as expected, the close relationship between intensities of paired spot duplicates on single arrays hybridized with unamplified or amplified cDNA respectively. Panel B shows the relationship between intensities of corresponding spots on two microarrays each hybridized with independent preparations of unamplified cDNA (“Dir1” and “Dir2”). The small degree of scatter, as expected, demonstrates the reproducibility of abundance measurements in independent preparations of unamplified cDNA. The critical comparisons are shown in Panels D and E. representative of numerous experiments, plotting corresponding spot intensities on separate microarrays hybridized to samples of cDNA amplified independently from differing quantities of RNA. The close relationship between hybridization intensities obtained independently between samples amplified from 1 and 10 ng quantities of RNA clearly establishes that abundance relationships in amplified cDNA are reproducible despite extensive exponential expansion of the original templates. Although somewhat more scatter is evident in panel E comparing results from amplification of 10 pg to those from 10 ng, dispersion remained a modest fraction of intensity as measured by mean relative error.

Differential Hybridization Intensity Patterns from Amplified and Unamplified Targets are Similar

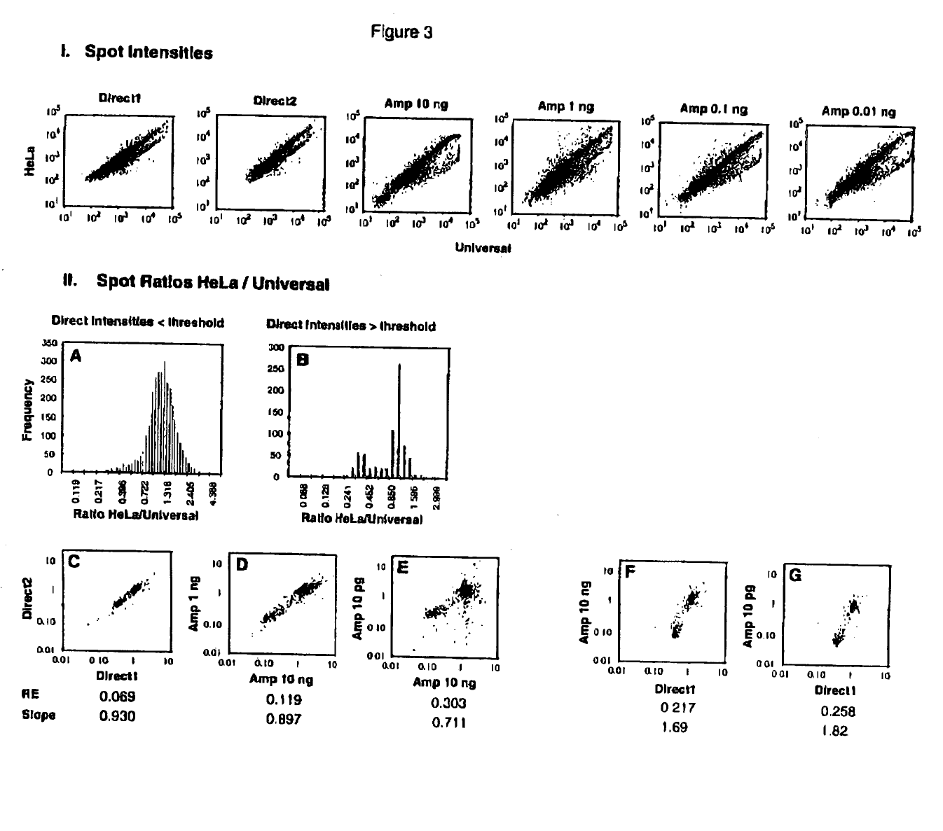

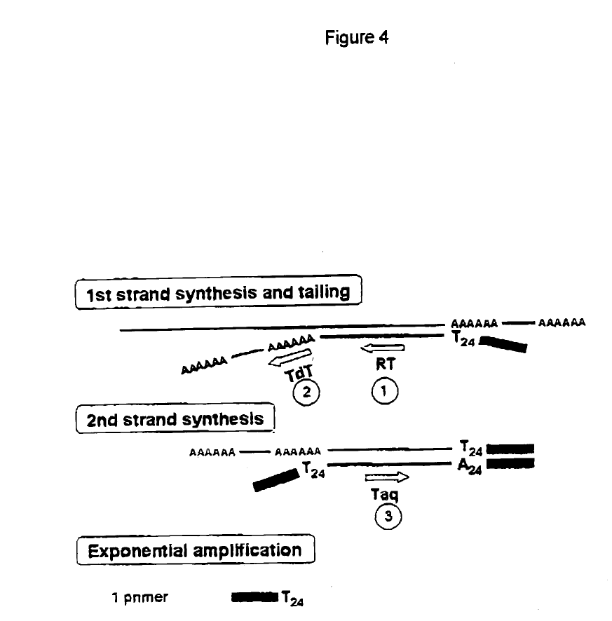

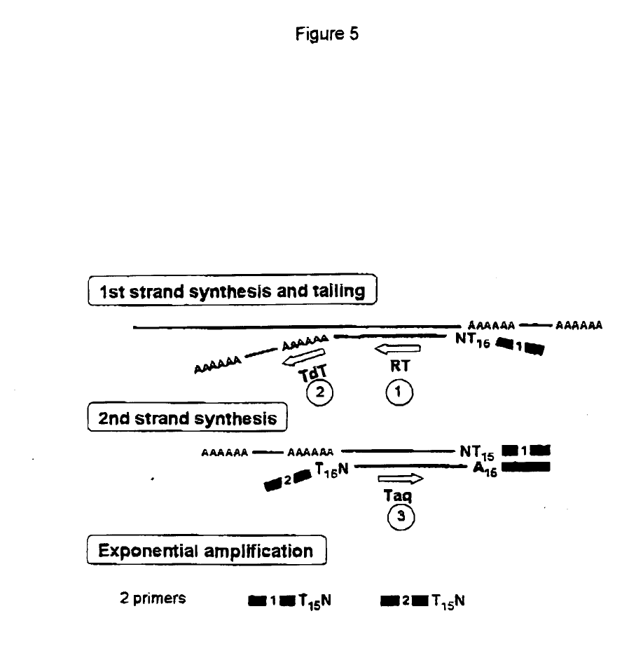

In succeeding analyses, we assessed transcript differences between HeLa cell and Reference RNA, and compared results from globally amplified cDNA with those from unamplified cDNA. In FIG. 3-I, each plot compares spot intensities on microarrays hybridized with differentially labelled HeLa and Universal cDNAs. The nature of each cDNA pair is indicated over each plot. As expected for differing RNA sources, dispersion was significantly greater than that observed with replicate determinations on a single RNA source (FIG. 2B). Strikingly, whether obtained with unamplified or amplified cDNA, each plot displays a similar pattern, indicative of a main population of spots illuminated to similar intensities by both targets, and a secondary population below the diagonal in which hybridization with the HeLa target is less intense than labelling with the Universal target. This is indeed the result expected from a comparison of RNA from a single cell type with RNA representative of a spectrum of cell types. Significantly, the same pattern was observed with cDNA targets amplified from RNA amounts ranging from 10 ng down to 10 pg.

Amplified and Unamplified Targets Yield Similar Hybridization Ratios for Individual Transcripts

Although the intensity profiles in FIG. 3-I suggested that abundance relationships in the amplified targets were similar to those in the unamplified targets, this point remained to be demonstrated directly. One way of testing for similarity would be to examine the correspondence between ratios of HeLa to Universal hybridization intensities for each spot.

Before performing the comparisons, it was necessary to separate meaningful hybridization signals from noise. A rough indication of the usable intensity range is evident in FIG. 3-I, where the underexpressed subpopulation is resolved only above relative Universal hybridization intensities of about 1000. An additional, more objective criterion is available by virtue of the presence on the arrays of 804 control spots containing either no cDNA (SSC hybridization buffer) or irrelevant sequence (PCR primer artifact or transcripts of plant (Arabidopsis) origin). We made use of these by sorting Cy3-Cy5 pairs in descending order of intensity and excluding data below the intensity level of the 2nd instance of a blank control. The cutoff identified by this heuristic occurred in all data sets near Universal spot intensities of 1000, and excluded approximately 75% of the arrayed probes. The ratios of HeLa to Universal hybridization intensities for each spot were next plotted as frequency histograms separately for the included and excluded populations. As shown in FIG. 3-II panel B, the high intensity data set segregated into two distinct populations. The main population consisted of spots clustering near a ratio of 1. The second, smaller population clustered near a ratio of 0.3. In distinct contrast, no such subpopulation was resolved in the low intensity population. This difference provided the rationale for confining further attention to the high intensity population comprising about 25% of the human cDNAs on the array.

In FIG. 3-II, a series of comparisons of HeLa to Universal hybridization intensity ratios obtained in different settings are shown in the bottom row. In panel C, the ratios obtained in two independent experiments from unamplified cDNA correlate quite closely. Moreover, the plot again indicates the subdivision of the points into a majority set clustering near a ratio of 1, and a subset clustered near 0.3. When cDNA amplified from 1 and 10 ng (panel D), or 10 pg and 10 ng (panel E) starting amounts of RNA were compared, the same pattern was observed, revealing again the same 2 populations and indicating again the reproducibility of abundance relationships in independent amplifications.

To test decisively for preservation of abundance in the amplified samples, we directly compared hybridization ratios obtained with unamplified and amplified cDNA targets on independent arrays. These results, shown in FIG. 3-II, panels F and G, confirm that ratios observed with amplified and unamplified targets are indeed closely correlated, even after amplification from as little as 10 pg of total RNA the outcome formally establishes that abundance relationships are substantially retained during extensive exponential amplification from the original RNA templates.

Amplified Targets Identify the Same Subsets of Over- and Underexpressed Transcripts as Unamplified Targets

We next asked how accurately differentially expressed genes would be detected using exponentially amplified cDNA targets. Transcripts were first identified whose representation in HeLa cell RNA differed by a factor of two or more from that in Universal RNA by hybridization of unamplified, differentially labelled cDNA to our microarrays. As detailed in Table 2, 84 such transcripts were identified that differed by at least two-fold on at least 3 of 4 replicate spots on duplicate microarrays. Of these, 65 (77%) were also detected as differentially expressed (>2-fold) on 6 or more of 8 replicate spots using targets amplified from 10000, 1000, 100 or 10 pg of total RNA. 19 transcripts, which were differentially represented in unamplified cDNA, were not detected as differentially expressed when amplified targets were used. Of these, 18 were not hybridized above the cutoff intensity by the amplified targets. Notably, on the single array hybridized with amplified cDNA from 10 pg of RNA, 70 of the 84 differences were detected on both replica spots. Conversely, 17 differentially expressed genes were detected in amplified targets that were not similarly detected in unamplified targets. Of these, 10 were hybridized below the intensity threshold by the unamplified targets, while 7 had expression ratios in unamplified cDNA under the 2-fold filter.

Materials & Methods

RNA

HeLa cells were grown in Dulbecco's media H2l with 100 mg/L each of penicillin G potassium and streptomycin sulfate. Cells were split when they reached confluence using phosphate-buffered saline (PBS), without calcium chloride and without magnesium chloride, and trypsin-EDTA (0.05% trypsin, 0.53 mM EDTA*4Na, Life Technologies). The total RNA was isolated using the RNeasy® Mini Kit (Qiagen), following the manufacturer's standard protocol for the isolation of total RNA from animal cells. RNA was eluted from the RNeasy® column with 60 μl RNase-free water (2×30 μl). The concentration was determined by spectrophotometer (Abs.260) and the integrity analyzed using the Agilent 2100 Bioanalyzer (Agilent Technologies).

Universal Reference RNA was obtained from Stratagene. This RNA is a pool of total RNA from 10 human cell lines (adenocarcinoma, mammary gland; hepatoblastoma, liver; adenocarcinoma, cervix; embryonal carcinoma, testis, glioblastoma, brain; melanoma; liposarcoma; histiocytic lymphoma, macrophage, histocyte; lymphoblastic leukemia, T lymphoblast; plasmacytoma, myeloma, B lymphocyte). The Universal Human Reference RNA (200 μg per tube) was provided in a solution of 70% ethanol and 0.1 M sodium acetate. The RNA was pelleted, washed and resuspended to a final volume of 2 μg/μl in RNase free water.

Preparation of Unamplified cDNA Target

For each labeling reaction, 10 μg of RNA was used. Reaction volumes were 40 μl in total. The reaction buffer contained 1× SuperScript II First Strand buffer (Life Technologies), 150 pmol of a modified oligo dT primer (5′-T₂₀VN, Cortec, Kingston, ON, Canada), 0.5 mM each of dATP, dGTP, and dTTP (Amersham Pharmacia Biotech), 0.05 mM dCTP (Amersham Pharmacia Biotech), 0.025 mM of either Cyanine 3 or Cyanine 5 dCTP (PE/NEN), and 0.01 mM DTT (Life Technologies). The labeling reaction was heated to 65° C. for 2 minutes to denature the RNA and then cooled to 42° C. Once a stable temperature of 42° C. was achieved, 2 μl of SuperScript II Reverse Transcriptase (Life Technologies) was added. Labelling reactions were allowed to run for 2 hours. After labeling, the tubes were centrifuged to pull down any condensate and then tubes were placed on ice. 4 μl of 50 mM EDTA (pH 8.0) and 2 μl of 10N NaOH was added to stop the reaction and allow for RNA hydrolysis. RNA was hydrolyzed at 65° C. for 20 minutes. After hydrolysis, the pH was returned to neutral by the addition of 5 M acetic acid. Labelled cDNA was then purified using Amicon PCR clean up columns (Amicon, Millipore). After purification, samples were ready for hybridization.

Preparation of Amplified cDNA Target

RNA in 0.5 μl H₂O, or as in Table 1 a single micromanipulated cell, was added to 4 μl first strand buffer (TrisHCl 50 mM pH 8.3, KCl 75 mM, MgCl₂ 3 mM, NP-40 0.5%, DTT 1 mM, acetylated BSA 100 μg/ml, RNA guard (Amersham/Pharmacia) 2.9 μl/100 μl, Prime Rnase Inhibitor (3 Prime 5 Prime Inc.) 0.1 μl/100 μl, fresh dNTPs 10 μM) in a 200 μl PCR tube. After heating to 65° C. for 90 sec, the tube was cooled to 50° C. and reverse transcription initiated by addition of 0.5 μl (100 U) SuperScript II (Invitrogen)) and 0.2 μl SR-T24 primer (5′ GTTAACTCGAGAATTC(T)24 ¹⁴), to a reaction concentration of 0.00245 ODU/ml, 3.5 nM). After 15 min the reaction was terminated by heating to 70° C. for 10 min. RNAse H 1.0 μl (Amersham/Pharmacia) and MgCl₂ 0.7 μl, (75 mM, combined final [Mg⁺⁺] 9.4 mM) were added and RNA digested at 37° C. for 15 min. The cDNA strands were tailed by addition of 6.5 μl 2× tailing buffer (Roche/Boehringer Mannheim, final reaction concentrations TrisHCl 25 mM, K cacodylate 200 mM pH 6.6, CoCl₂ 1.5 mM) containing dATP to a final reaction concentration of 750 μM and terminal deoxynucleotidyl transferase (Roche/Boehringer Mannheim) 0.5 μl (25U), and incubated at 37° C. for 15 min. Tailing was stopped by heating to 65° C. for 10 min. 4 μl of he reaction was added to each of 3 tubes containing 15 μl polymerase buffer (TrisHCl 10 mM pH 8.3, KCl 50 mM, MgCl₂ 1.5 mM, BSA 100 μg/ml and Triton X-100 0.05%), dNTPs 0.875 mM and SR-T24 primer 7.5 ODU/ml, 11 μM. The resulting mixture contained totals of TrisHCl 13 mM pH8.3, MgCl₂ 2.5 mM, and residual CoCl₂ 0.3 mM. To generate the second cDNA strands, the mixture was overlaid with mineral oil and 2U Taq+0.05 U Pfu (Stratagene) polymerases were added at 94° C. Primer was annealed at 50° C. for 2 min followed by 2 minutes of extension at 72° C. After amplification through 30 additional cycles (94° C. 15 sec; 60° C. 30 sec. 72° C. 2 mm), 1 μl from each of the 3 tubes was pooled and 0.2 μl added to 18 μl of polymerase buffer containing dNTPs 0.2 mM and primer at 1.8 ODU/ml, 2.5 μM. The mixture was overlaid with mineral oil, Taq and Pfu polymerases were added at 94° C., and an additional 35 cycles of amplification were performed. Amplified stock cDNA was stored at −20° C.

For generation of dye-coupled cDNA, 1 μl of 1:100 diluted amplified stock cDNA was added to 98 μl polymerase buffer containing 0.5 ODU/ml, 0.7 RM SR-T24 primer, 0.1 mM dTTP, and 0.2 mM each of dCTP, dGTP, dATP and amino allyl dUTP (Sigma). Taq polymerase, 2 U, was added at 94° C., and the mixture amplified through 35 cycles (94° C. 15 sec; 60° C. 30 sec, 72° C. 1 min). Aminoallyl cDNA (typically 5 μg) was purified on a Microcon-30 column (Millipore) according to directions, concentrated to 2-3 μl by centrifugation under vacuum, and 1 μl was added to 5 μl 0.1 M NaHCO3, pH 9.0. The contents of 1 vial of Cy3 or Cy5 monofunctional reactive dye (Amersham/Pharmacia) were dissolved in 45 μl DMSO. Aminoallyl cDNA, 5 μl, was mixed with 5 μl of dye and the tube wrapped in foil to exclude light and incubated 30 min at room temperature. Labelled cDNA was isolated using a High Pure PCR purification kit (Boehringer Mannheim) according to directions and the eluate concentrated to 5-7 4 μl by vacuum centrifugation. Labelling efficiency was measured in some samples by optical density at 260 nm and 550 nm for Cy3 or 650 nm for Cy5. Arrays were hybridized with approximately 1 μg each of HeLa and Universal cDNA.

Hybridization of Microarrays

Hybridization buffer (DIG Easy Hyb (Roche) containing 50 μg each of yeast tRNA (Invitrogen) and calf thymus DNA (Sigma) per 100 μl) was added to concentrated Cy3 and Cy5 labelled cDNA to a total volume of 50 μl, heated to 65° C. for 2 min. and pipetted onto a 24×30 mm coverslip. A microarray slide was lowered onto The coverslip, inverted, placed in a closed, water-containing plastic hybridization chamber and incubated on a level surface for 16 hr at 42° C. in a covered water bath. The coverslip was removed by immersion of the array in 1× SSC. The array was washed 5 times for 5 min at room temperature in 0.1× SSC/0.1% SDS with agitation, rinsed 3 times in 0.1× SSC, dried by centrifugation, and scanned with minimum delay

Microarrays

Arrays were printed using a Virtek ChipWriter (Virtek Vision, Waterloo) onto Coming CMT-GAPSII slides. Arrays consisted of 1486 distinct PCR-amplified human cDNAs, each in duplicate to give a total of 2972 spots. In addition, the arrays contained 256 spots of plant (Arabidopsis) cDNA and a further 548 blanks containing either SSC or PCR priming artifact.

Where spot intensities on different slides were directly compared, the slides were always from a single production run.

Image Acquisition and Quantitation

Arrays were scanned on a Packard Biochip ScanArray 4000XL. The Cyanine 3 and Cyanine 5 channels were balanced by eye with the ScanArray Software to ensure a similar dynamic range for the two channels. Final scans were at 10 μm resolution, and images for each channel were saved as separate 16-bit TIFF files. These were imported into GenePix (Axon) for quantitation.

Data Analysis

Raw intensity ratios (HeLa/Universal) were computed and normalized by dividing by the modal ratio. Raw Cy3 intensities were sorted in descending order and the ordinal position of the second instance of a control spot (Arabidopsis, SSC or empty PCR reaction) was recorded. Cy5 intensities were similarly sorted. Intensities at and below the second instance of a control spot (Arabidopsis, SSC or empty PCR reaction) were excluded from further analysis, using the Cy3 or Cy5 sort that yielded the greater number of spots above cutoff. This strategy excluded a few spots, which might have intensities above threshold in one channel but below threshold in the other. Such instances could be valuable in a biological context but for simplicity were excluded from the present statistical analysis. Spots with normalized intensity ratios between 0.9 and 1.1 were identified and their average Cy3 and Cy5 intensities (C3 and C5) computed. Each array spot intensity (1) was then normalized to [I/Cx]×10000.

The relative error between 2 measurements V was defined as the ratio of their standard deviation to their mean. This metric reduces algebraically to |V1−V2|/(V1+V2)

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Enhancement of priming specificity for mRNA in the reverse
transcription reaction by initiating priming only at the
outset of the reverse transcription step.

| | Number of clones | | |
|---|---|---|---|
| Starting | Polyadenylation signal | | |
| condition | Present | Absent | Total |
| Cold | 10 | 18 | 28 |
| Hot | 31 | 12 | 43 |
| Legend: | | | |
| CDNA was generated by global RT-PCR from single micromanipulated cells from a human leukemia cell line, according to the procedure detailed in Experimental Protocol. For 1 of the cells, the 1^{st} strand buffer already contained the SR-T24 primer (“cold priming”). For the other cell, primer was added only at the time of addition of SuperScriptll reverse transcriptase at 50° C.(“hot priming”). For both, reverse | | | |
| # transcription was preformed at 50° C. Amplified cDNA was cloned into plasmids, transformed bacterial clones were randomly selected, plasmids isolated and inserts sequenced through their entire length. In clod priming conditions, only 36% (10/28) of inserts contained a polyadenylation signal, while 72% contained polyadenylation signals after hot priming (P < 0.005, 2 × 2 X² test). None of the inserts consisted | | | |
| # of primer artifact. Average cDNA insert length, excluding primer sites, was 360 b | | | |

TABLE 2
Microarrayed probes differing by 2-fold or more
in HeLa and Universal cDNA targets.

| | |
|---|---|
| 1468 | total human EST probes on micrarray |
| 350 | probes above instensity threshold in HeLa or Universal targets |
| 101 | probes differing >=2-fold between HeLa and Universal targets |
| 19 | >2-fold only in unamplified target |
| 18 | <intensity threshold in amplified target |
| 1 | <2-fold in amplified target |
| 65 | <2-fold in both unamplified and amplified targets |
| 17 | >2-fold only in amplified target |
| 10 | <intensity threshold in unamplified target |
| 7 | <2-fold in unamplified target |

Legend:
Listed are probes differing at least 3 of 4 spots in 2 tests of unamplified targets, and in at icast 6 of 8 spots in 4 tests of amplified targets. Of 101 probes that differed, 3 were overrepresented and 98 underrepresented in HeLa relative to Universal cDNA. In unamplified targets, 84 (65 + 19) probes differed by at least 2-fold, of which 77% (65/84) were detectable in amplified targets.

References

1. Wang, E., Miller, L. D., Ohnmacht, G. A., Liu, E. T. & Marincola, F. M. High-fidelity mRNA amplification for gene profiling Nat Biotechnol 18, 457-9. (2000).

2. Baugh, L. R., Hill, A. A., Brown, E. L. & Hunter, C. P. Quantitative analysis of mRNA amplification by in vitro transcription. Nucleic Acids Research 29, E29/1-9 (2001).

3. Phillips, J. K. & Lipski, J. Single-cell RT-PCR as a tool To study gene expression in central and peripheral autonomic neurones. Auton Neurosci 86, 1-12. (2000).

4. Freeman, T. C., Lee, K. & Richardson, P. J. Analysis of gene expression in single cells. Curr Opin Biotechnol 10, 579-82. (1999).

5. Dixon, A. K., Richardson, P. J., Pinnock, R. D. & Lee, K. Gene-expression analysis at the single-cell level. Trends Pharmacol Sci 21, 65-70. (2000).

6. Brady, G., Barbara, M. & Iscove, N. N. Representative in vitro cDNA amplification from individual hemopoietic cells and colonies. Methods Molec. Cell Biol. 2, 17-25 (1990).

7. Brady, G. & Iscove, N. N. in Methods in Enzymology (eds. Wassarman, P. M. & DePamphilis, M. L.) 225:611-623 (Academic Press, San Diego, 1993).

8. Brady, G. Expression profiling of single mammalian cells—small is beautiful. Yeast 17, 211-7. (2000).

9. Brady, G., et al. Analysis of gene expression in a complex differentiation hierarchy by global amplification of cDNA from single cells. Current Biology 5, 909-922 (1995).

10. Billia, F., Barbara, M., McEwen, J., Trevisan, M. & Iscove, N. N. Resolution of pluripotential intermediates in murine hematopoietic differentiation by global cdna amplification from single cells: confirmation of assignments by expression profiling of cytokine receptor transcripts. Blood 97, 2257-2268 (2001).

11. Brail, L. H., et al. Gene expression in individual cells: Analysis using global single cell reverse transcription polymerase chain reaction (GSC RT-PCR). Mutat. Res. 406, 45-54 (1999).

12. Cano-Gauci, D. F., et al. In vitro cDNA amplification from individual intestinal crypts: A novel approach to the study of differential gene expression along the crypt-villus axis. Exp. Cell Res 208, 344349 (1993).

13. Trumper, L. H., et al. Single-cell analysis of Reed-Sternberg cells: Molecular heterogeneity of gene expression and p53 mutations. Blood 81, 3097-3115 (1993).

14. Baugh, L. R., Hill, A. A., Brown, E. L. & Hunter, C. P. Quantitative analysis of mRNA amplification by in vitro transcription. Nucleic Acids Research 29, E29/1-9 (2001).

15- Phillips, J. K. & Lipski, J. Single-cell RT-PCR as a tool to study gene expression in central and peripheral autonomic neurones. Auton. Neurosci. 86, 1-12. (2000).

16. Freeman, T. C., Lee, K. & Richardson, P. J. Analysis of gene expression in single cells. Curr. Opin. Biotechnol. 10, 579-582. (1999).

17. Dixon, A. K., Richardson, P. J., Pinnock, R. D. & Lee, K. Gene-expression analysis at the single-cell level. Trends Pharmacol. Sci. 21, 65-70. (2000).

We claim:

1. A process for generating nucleic acid targets or probes comprising the steps of:
(A) providing an RNA preparation that comprises polyadenylated mRNA;
(B) providing a first oligonucleotide primer that comprises
(1) a first segment containing a unique sequence; and
(2) a second segment being substantially complementary to the polyadenylated mRNA and capable of template-dependent first strand synthesis;
(C) contacting the mRNA with the first primer to generate by DNA polymerase or reverse transcriptase reaction from the polyadenylated mRNA, DNA strands that are substantially complementary to the polyadenylated mRNA;
(D) adding a polynucleotide tail to the 3′ end of the DNA strands whereby the DNA strands have a first portion that is complementary to the polyadenylated mRNA and a tail portion;
(E) providing a second oligonucleotide primer that comprises
(1) a first segment containing a unique sequence; and
(2) a second segment being substantially complementary to the tail portion of the DNA strand and capable of Template-dependent second strand synthesis;
(F) contacting the complementary DNA with the second primer to generate by a DNA polymerase reaction from the tailed DNA, DNA strands that are substantially complementary to the tailed DNA; and
(G) contacting the DNA strands with the first primer and the second primer to amplify exponentially, by at least 1000-fold, the DNA strands by repetitive cycles of thermal denaturation, annealing and DNA polymerase reaction, to produce the targets or probes.

2. The process of claim 1, wherein the RNA is isolated from a biological sample selected from the group consisting of a body fluid, stool a single cell, dissected tissue, microdissected tissue, a tissue subregion, a tissue biopsy sample, cells recovered from body fluids or from the body in aspirates or scrapings or washings, a cell sorted population and a cell culture.

3. The process of claim 1, wherein the RNA is isolated from a cell or tissue selected from the group consisting of brain, liver, heart, kidney, lung, spleen, eye, retina, bone, lymph node, endocrine, endocrine gland, secretory gland, reproductive organ, blood, marrow, bone, cartilage, muscle, fat, connective tissue, nerve, vascular tissue, skin, hair, epithelial and mesothelial structures or surfaces

4. The process of claim 1 wherein the RNA is isolated from a cell or tissue selected from the group consisting of non-embryonic cell or tissue, embryonic, pathological and tumorigenic.

5. The process of claim 1, wherein the amount of RNA present is less than 10 ng.

6. The process of claim 1, wherein the polynucleotide tail is selected from the group consisting of poly(A), poly(G), poly(C) or poly(T).

7. The process of claim 1, wherein the first and second oligonucleotide primers are identical.

8. The process of claim 1, wherein the first and second oligonucleotide primers are different.

9. The process of claim 1, wherein the first and second oligonucleotide primers provide for non-directional amplification of the polyadenylated mRNA.

10. The process of claim 1, wherein the first and second oligonucleotide primers are anchored primers.

11. The process of claim 1, wherein the first and second oligonucleotide primers provide for directional amplification of the polyadenylated mRNA.

12. The process of claim 1, wherein the gene expression monitoring system is selected from the group comprising DNA array, biochip, DNA chip, DNA microarray, gene array, real time quantitative PCR.

13. The process of claim 1, further comprising digesting any residual RNA remaining after step C by the addition of RNAse H.

14. The process of claim 1, wherein the initial reverse transcription reaction occurs between about 30° C. to about 100° C. and the DNA polymerase reaction occurs between about 23° C. to about 100° C.

15. The process of claim 1, wherein the amplification comprises at least 20 cycles of denaturation, annealing, and DNA polymerase reaction.

16. The process of claim 1, wherein the amplification cycles occurs between 23° C. to 100° C., denaturation occurs between 90° C. to 100° C., annealing occurs between 37° C. to about 75° C., and DNA polymerase reaction occurs between about 37° C. to about 80° C.

17. The processes of claim 1, wherein one or more nucleotides that are covalently coupled to fluorochromes are incorporated during the repetitive cycles of thermal denaturation, annealing and DNA polymerase reaction to directly generate fluorochrome-coupled nucleic acid targets or probes.

18. The process of claim 1, wherein one or more nucleotides containing reactive side groups are incorporated during the repetitive cycles of thermal denaturation, annealing and DNA polymerase reaction, to directly generate reactive-side-group-coupled nucleic acid target or probes.

19. The process of claim 18 wherein the reactive-side-group-coupled nucleic acid targets or probes are modified by the addition of fluorochrome.

20. The method of claim 1, wherein the polyadenylated mRNA comprises between 0.1 picograms and 10 ng of RNA.

21. The method of claim 1, wherein the polyadenylated mRNA is obtained from a single cell

22. The method of claim 1, wherein the 200 to 600 nucleotides at the 3′ terminus of the mRNA are amplified.

23. A nucleic acid target produced by the process of claim 1.

24. A nucleic acid probe produced by the process of claim 1.

25. The process of claim 1, wherein the process amplifies targets to generate cDNA libraries and the representation of particular gene transcripts is measured.

26. A kit for generating nucleic acid probes for use in gene expression monitoring systems, wherein the kit comprises a reverse transcriptase, a DNA polymerase, a terminal deoxynucleotidyl transferase and oligonucleotide primers

27. A kit for generating nucleic acid targets for use in gene expression monitoring systems, wherein the kit comprises a reverse transcriptase, a DNA polymerase and oligonucleotide primers.
